(19) United States
(12) Patent Application Publication
(10) Pub. No.: US 2015/0185339 A1
(43) Pub. Date: Jul. 2, 2015

(54) MULTIPLEXABLE EMISSION TOMOGRAPHY

(71) Applicants: Eduardo M. Lage, Boston, MA (US); Joaquin L. Herraiz, Brookline, MA (US); Vicente J. Parot, Cambridge, MA (US)

(72) Inventors: Eduardo M. Lage, Boston, MA (US); Joaquin L. Herraiz, Brookline, MA (US); Vicente J. Parot, Cambridge, MA (US)

(21) Appl. No.: 14/396,805

(22) PCT Filed: Apr. 30, 2013
(86) PCT No.: PCT/US13/38846

§ 371 Date: Oct. 24, 2014

Related U.S. Application Data
(60) Provisional application No. 61/640,292, filed on Apr. 30, 2012.

(51) Int. Cl. G01T 1/29; A61B 6/00; A61B 6/03

(52) U.S. Cl. CPC G01T 1/2985; A61B 6/037; A61B 6/5205; A61B 6/4258

(57) ABSTRACT

A method and system for acquiring a series of medical images during a common imaging process, includes a plurality of detectors configured to be arranged to acquire gamma rays emitted from a subject as a result of multiple radiotracers administered to the subject and communicate signals corresponding to acquired gamma rays. A data processing system is configured to receive the signals from the plurality of detectors and identify temporal information and energy information of photons of the acquired gamma rays. A reconstruction system is configured to receive the signals, the temporal information, and the energy information from the data processing system and reconstruct therefrom a series of medical images of the subject, wherein at least one of the images in the series of medical images corresponds to only to information acquired from gamma rays emitted as a result of a given one of the multiple radiotracers.

### CROSS-REFERENCE TO RELATED APPLICATIONS

This application is based on, claims priority to, and incorporates herein by reference in its entirety, U.S. Provisional Application Ser. No. 61/640,292, filed Apr. 30, 2012, and entitled “MULTIPLEXABLE EMISSION TOMOGRAPHY”

### STATEMENT REGARDING FEDERALLY SPONSORED RESEARCH

N/A

### BACKGROUND OF THE INVENTION

The present invention relates to systems and methods for emission tomography and, more particularly, to systems and methods for multiplexable emission tomography that allow researchers and clinicians to acquire data from multiple, distinguishable sources using emission tomography.

There are a variety of emission tomography imaging systems and methods. For example, single photon emission computed tomography (SPECT) and positron emission tomography (PET) are two clinically important examples. Both PET and SPECT, and other various emission tomography systems and methods, utilize a radionuclide, typically bonded or otherwise coupled to another compound, often a targeting compound referred to broadly as a radiopharmaceutical.

Positrons are positively charged electrons and are emitted by radionuclides that have been prepared using a cyclotron or other device. The radionuclides most often employed in diagnostic imaging are fluorine-18 (¹⁸F), carbon-11 (¹¹C), nitrogen-13 (¹³N), and oxygen-15 (¹⁵O). Furthermore, there are other radionuclides like ⁶⁰Cu, ¹²⁴I, ⁸²Rb, ⁸⁶Y, ^{94m}TC or ⁷⁶Br, which have also been used successfully in molecular imaging. These radionuclides are employed as radioactive tracers called “radiopharmaceuticals” by incorporating them into substances, such as glucose or carbon dioxide. The radiopharmaceuticals are administered to the patient and become involved in such processes as blood flow, fatty acid and glucose metabolism, and protein synthesis.

The radiopharmaceutical is injected into the subject to accumulate in or otherwise target an area or organs of interest in the subject. By measuring or identifying photons emitted from the area or organs of interest by the accumulated or targeted radiopharmaceutical, clinically useful anatomical and, more importantly, biological and physiological information can be obtained.

PET, SPECT, variations thereon, and, generally, emission-based tomographic systems and methods share similarities in their use of radioactive tracer material and detection of gamma rays. In contrast with PET, however, the tracer used in SPECT emits gamma radiation that is measured directly, whereas PET tracer emits positrons that annihilate with electrons, causing two gamma photons to be emitted in opposite directions.

Thus, with respect to SPECT, tracer distribution is measured, at a prescribed time following marker injection, using a gamma camera that is positioned adjacent the portion of a patient's body that includes the area or organ to be imaged. During an imaging period with the camera supported in a single position and the patient remaining as still as possible, the camera detects photon emissions and can create a two-dimensional projection view of the organ corresponding to the camera position. Most SPECT gamma imaging procedures used to generate tomographic images require a plurality of such emission projection images, each image taken by positioning the gamma camera at different view angles about an imaging axis. Multi-isotope, multiplexable imaging is readily compatible with SPECT systems, since several radiotracers can be labeled with a different gamma emitter radionuclide that emits a characteristic gamma-ray with a known energy. Signal separation can be done based on the energy differences of the detected gamma rays.

With respect to PET, as the injected radionuclide decays, it emits positrons. The positrons travel a very short distance before they encounter an electron and, when this occurs, the positrons are annihilated and converted into two photons, or gamma rays. This annihilation event is characterized by two features that are pertinent to PET imaging. Namely, each gamma ray has an energy of 511 keV and the two gamma rays are directed in substantially opposite directions. An image is created by determining the number of such annihilation events at each location within the scanner's field of view.

To create such an image, one or more rings of detectors are positioned to encircle the patient and detect photons within a predefined energy range which includes 511 keV. Coincidence detection circuits connected to the detectors record only those photons that are detected simultaneously by two detectors located on opposite sides of the patient. The number of such simultaneous events indicates the number of positron annihilations that occurred along a line joining the two opposing detectors. Within a few minutes, hundreds of millions of events can be recorded to indicate the number of annihilations along lines joining pairs of detectors in the ring. These numbers are employed to reconstruct an image using well-known computed tomography techniques. However, current technology permits only one radiotracer to be imaged at a time using PET, because positron-electron annihilation products from different positron-emitting radionuclides are indistinguishable in terms of energy.

PET, SPECT, and other emission tomography systems and methods provide highly-valuable biological and physiological information. That is, compared, for example, with other common imaging modalities, such as magnetic resonance imaging (MRI) or computed tomography (CT), PET, SPECT, and other emission tomography systems and methods provide superior information about the underlying operation and function of the tissue(s) or organ(s) being studied.

The use of a radiotracer with imaging emission tomography modalities, such as PET and SPECT, enables the acquisition of this superior biological and physiological information when compared with other common clinical imaging modalities. The radiotracer also presents a fundamental limitation on the amount and type of clinical information that can be acquired. Specifically, as described above, PET and SPECT systems rely on particular, anticipated, characteristics of the emitted photons. That is, PET, SPECT, and other emission tomography systems that utilize radiotracers employ specialized hardware (e.g., ring of gamma detectors with individual detectors arranged in perfect opposition, collimators with a specific thickness) and software (e.g., coincidence detection algorithms that consider only photons with energy levels of 511 keV±ΔE, being ΔE a function of the energy resolution capabilities of the system) tailored to the particular, anticipated, characteristics of the emitted photons. Additionally, such emission tomography processes are also limited by the particular radiopharmaceutical employed and the radiopharmaceutical's particular target. That is, traditional emission tomography system and methods are generally limited to investigating or considering one biological or physiological target of investigation because the selected radiopharmaceutical will, primarily, target only one biological or physiological target at a time (e.g., one of blood flow, fatty acid metabolism, glucose metabolism, protein synthesis, or the like).

For example, one very-popular PET radiopharmaceutical is 18F-fluorodeoxyglucose (FDG), which is a marker for hexokinase activity, which is the rate-limiting step in glucose metabolism. Since most cancerous cells exhibit markedly increased FDG uptake as compared to normal cells, FDG PET is a fairly-general tool for cancer imaging. As such, FDG PET has gained wide acceptance for cancer diagnosis, staging, and evaluating recurrent or residual disease following therapy. Though a powerful agent, FDG has several limitations. For example, FDG uptake is not specific to neoplastic disease. Also, inflammatory responses can lead to false-positive findings. Furthermore, certain malignancies, such as some neuroendocrine tumors, gastric or prostate cancer as well as some small pulmonary nodules, are not particularly FDG avid, leading to low sensitivity for FDG PET. Additionally, tumors are complex and cannot be fully characterized by one single parameter, such as increased glucose metabolism. Other relevant characteristics like perfusion, hypoxia (low oxygenation level), apoptosis (cell death), and receptors are of great interest for a correct diagnosis, staging, treatment and evaluation of the response to the therapy. Each of these characteristics can be measured currently with molecular imaging techniques (PET) by using separated acquisitions. Accordingly, though FDG is a clinically advantageous selection for many PET imaging protocols, it has a variety of drawbacks and, currently, there is no mechanism for controlling or mitigating such drawbacks, short of conducting additional PET imaging acquisitions using serial selections of radiopharmaceuticals, which is clinically impractical, costly, and, often, otherwise inadvisable.

Although multiplexed imaging is possible with SPECT systems, their low sensitivity (100 to 1000 times lower than PET), low spatial resolution (between 2 to 3 times lower than PET) and the fact that SPECT is not inherently a quantitative technique (as it is PET), limits the usefulness of multiplexed SPECT imaging.

Therefore, it would be desirable to have a system and method for medical imaging that does not suffer from the above limitations.

### SUMMARY OF THE INVENTION

The present invention overcomes the aforementioned drawbacks by providing a system and method for multiplexed emission tomography (MET) that enables the use of multiple radiopharmaceuticals during an emission tomography imaging acquisition by enabling simultaneous tracking of temporal variations and discrimination of energy levels between acquired photons. Thus, the present invention provides systems, software, and methods for clinicians to perform a single emission tomography imaging acquisition using two or more, distinct, radiotracers and create distinct, but correlated images from the collected data that correspond to each of the administered radiotracers.

In accordance with one aspect of the present invention, an emission tomography system is disclosed for acquiring a series of medical images of a subject during a common imaging process using multiple radiotracers. The system includes a plurality of detectors configured to be arranged around he subject to acquire gamma rays emitted from the subject as a result of multiple radiotracers administered to the subject and communicate signals corresponding to acquired gamma rays. A data processing system is configured to receive the signals from the plurality of detectors and identify temporal information and energy information of photons of the acquired gamma rays. A reconstruction system is configured to receive the signals, the temporal information, and the energy information from the data processing system and reconstruct therefrom a series of medical images of the subject, wherein at least one of the images in the series of medical images corresponds only to information acquired from gamma rays emitted as a result of a given one of the multiple radiotracers.

In accordance with another aspect of the present invention, a method for acquiring a series of medical images of a subject is disclosed that includes administering to the subject at least two radiotracers selected to emit photons distinguishable in at least one of time and energy. The method also includes detecting photons emitted from the subject as a result of the at least two radiotracers administered to the subject, creating imaging data based on the detected photons, and processing the imaging data to identify at least one of temporal information and energy information associated with the detected photons. The method further includes sorting the imaging data into datasets distinguished by at least one of the temporal information and the energy information, wherein at least one dataset corresponds to only one of the at least two radiotracers, and reconstructing a series of medical images of the subject, wherein at least one of the images in the series of medical images corresponds to only one of the at least two radiotracers.

In accordance with yet another aspect of the invention, a method is provided for acquiring a series of medical images of a subject having been administered at least two radiotracers selected to emit photons distinguishable in at least one of time and energy. The method includes acquiring, during single imaging acquisition, photons emitted from the subject as a result of the at least two radiotracers administered to the subject, wherein the acquired photons are selected from a predetermined energy range. The method also includes creating, based on the acquired photons, imaging data sets, wherein each imaging data set is differentiated based on temporal information including coincidence events and energy information associated with the acquired photons. Furthermore, the method includes reconstructing a series of medical images of the subject from the imaging data sets, wherein at least one of the images in the series of medical images corresponds to only one of the at least two radiotracers.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic view of an emission tomography system in accordance with the present invention.

FIG. 2 is a schematic view of a positron emission tomography (PET) system adapted to operate as an emission tomography system in accordance with the present invention.

FIG. 3 is a flow chart setting forth the steps of an example of a method of using an emission tomography system in accordance with the present invention.

FIG. 4 is a flow chart setting forth the steps of an example of a method for reconstructing multiplexed emission tomography images in accordance with the present invention.

FIG. 5 is a flow chart setting forth the steps of an example of a method for building image data sets.

### DETAILED DESCRIPTION OF THE PREFERRED EMBODIMENT

The present invention recognizes that one of the great strengths of emission tomography, such as positron emission tomography (PET), is the ability to obtain images with high spatial resolution and sensitivity using any of a number of molecular or physiologic targets and different radiotracers. The development of new probes for imaging hypoxia, cell proliferation, blood flow, and numerous other molecular targets, which offers high potential for better diagnosis, characterization of disease, and image-guided personalized medicine. However, much of this potential remains unrealized because current technology permits only one radiotracer to be imaged at a time. Consequently, multiple scanning sessions need to be coordinated, and even scheduled on different days, to obtain complementary information from multiple radiotracers, which results in high costs, image alignment issues, and a long and onerous experience for the patient. As will be described, the present invention overcomes these drawbacks by providing a system and method for multiplexed emission tomography, thereby allow the imaging and distinguishing of multiple radiotracers simultaneously.

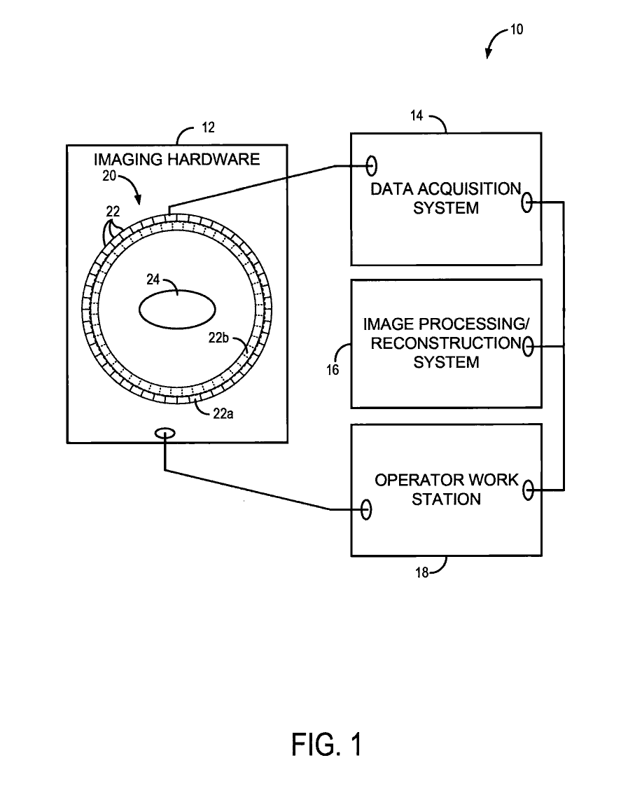

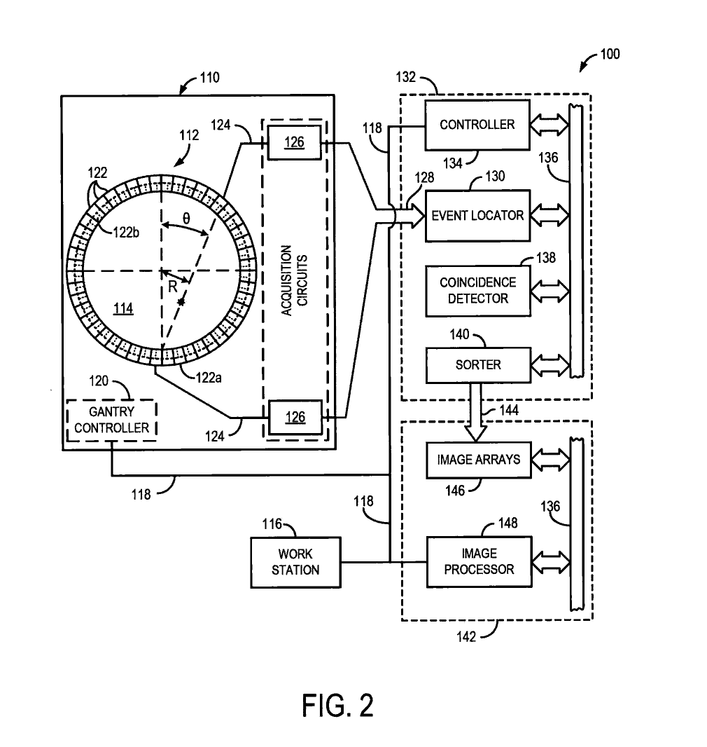

Referring particularly to FIG. 1, an emission tomography system 10 adapted for use as a multiplexed emission tomography (MET) system in accordance with the present invention is illustrated. As will be described, the emission tomography system may operate as a traditional positron emission tomography (PET) system, such as system 100 of FIG. 2, and, thus, include components of a PET system along with additional hardware and/or software to allow the system to operate as a MET system. Also, as will be described, it is contemplated that the system may represent another emission tomography system, such as a single photon emission computed tomography (SPECT) or other emission tomography system adapted to operate as a MET system.

As will be described, a MET system, such as a system 10, provides hardware and software that allows clinicians to acquire and process single or multiple “photon events” (two or more photons detected within a predetermined window), even simultaneously and at different detector elements, distinguish the energy information of each of the photons, and reconstruct images therefrom. As such, the present invention provides systems, software, and methods to obtain separated images with quantitative information about the bio-distribution of two or more, different, positron-labeled radiotracers in a single scanning session. That is, the present invention provides systems, software, and methods for clinicians to perform a single emission tomography imaging acquisition using two or more, distinct, radiotracers and create distinct, but correlated images from the collected data that correspond to each of the administered radiotracers.

Referring to FIG. 1, such a system 10 includes a plurality of subsystems, including imaging hardware 12, data acquisition systems 14, image processing/reconstruction systems 16, and an operator work station 18. The imaging hardware 12 includes a plurality of detectors 22, and associated hardware, to acquire photons emitted from a subject 24 arranged proximate to the detectors 22.

As described above, the system 10 is designed to image, in a single scanning session, the subject 24 after having been administered two or more (for example, “N”), different radiotracers. To do so, the system 10 is configured to create N distinct datasets, where at least one dataset corresponds to one of the administered radiotracers, and create a series of images, where at least one image may correspond to only data associated with one of the radiotracers.

As will be explained in further detail, the series (two or more) of desired radiotracers, each of them labeled with a different radionuclide, are introduced into the subject 24 using staggered or parallel injections. The order of the injections will depend on the physical characteristics of the radionuclides used to label the radiotracers and the biological behavior of the radiotracer. At a minimum, the characteristics of the radionuclides used to label the radiotracers will be such that the resulting photon events are distinguishable in at least one of time, number of photons emitted per decay, and energy. For example, one of the radiotracers may be labeled with a pure positron emitter radionuclide and others may be labeled with radionuclides that emit different radiation, for example, prompt gamma rays in cascade with a positron emission. The energy of the additional cascade gamma rays emitted by each of those radionuclides does not have to be different or distinguishable from the annihilation gamma rays (511 keV).

Thus, the detectors 22 are configured to collect a wide range of photon events to, thereby, allow the data acquisition system 14 to record multiple photon events and determine with certain precision the energy of each of the multiple photons comprising a coincidence event and their arrival time.

Such capabilities may be embodied in software and implemented by the data acquisition system 14 or, as illustrated, dedicated hardware may be employed to aid in the acquisition of the desired data and, more particularly, parsing the data into the N datasets based on the different criteria that distinguish between the administered radiotracers. Specifically, the detectors 22 may be formed as a ring of detectors arranged in a gantry, such as is commonly employed, for example in PET imaging systems. However, as will be described in further detail, the detectors 22 may include a single ring of detectors 22a, or may, optionally, include one or more additional energy-sensitive detectors 22b sharing a common time reference with the first detectors 22a. Other configurations, including different shapes, of detectors are contemplated, as the above-described and following description of a ring of detectors mounted on a gantry is for non-limiting, exemplary purposes. The present invention may be used with any variety of detector configurations.

Specifically, the system 10 may optionally include one, two, or more detector systems 22a and 22b that are specifically designed to acquire and process multiple photon events occurring simultaneously (within a time window) to obtain at least one of the energy, spatial, and temporal information of each of the detected photon events and reconstruct images therefrom.

For example, the first detectors 22a may be similar to traditional PET detectors and be tailored (via hardware and/or software) to identify photon coincidence events with photons having 511 keV of energy. In this regard, a first dataset (dataset A) is provided to the data acquisition system 13 corresponding to two-photon coincidence events coming from the radiotracers injected to the patient or subject 24 being studied. The energy of coincidence photons accepted in dataset A will be in a range that includes 511 keV.

The second ring of detectors 22b may be designed to acquire both alone or in collaboration with the first ring of detectors 22a, other n−1 datasets (dataset B . . . dataset n−1), apart or also with dataset A, that correspond to photon events distinguishable from the criteria used to create dataset A. Thus, the data in dataset A will include events in which two of the coincident photons are meeting a first criteria and data in datasets B . . . N−1 will include events in which other gamma rays were detected under a second, third, or N−1^{st} criteria.

As a non-limiting example, the first criteria may be common to positron annihilation (e.g., coincidence events at an energy of 511 keV) and the remaining criteria may be gamma rays received concurrently with the positron annihilations with energy coherent with the emission spectra of one of the secondary positron-gamma emitter isotopes injected.

As described above, the detectors may include, at least in some configurations, traditional PET detectors. Simultaneous dual-tracer PET imaging is difficult to perform because positron-electron annihilation products from different tracers are indistinguishable among them in terms of energy (511 keV) and the number of gamma rays generated (two). As such, some have attempted to perform simultaneous dual-tracer PET imaging, whereby the time delay between the administration of the two radiotracers is used to differentiate data corresponding to a particular radiotracer. However, such efforts are cumbersome and require extensive scanning durations that may not be feasible for some patients and, particularly, patients suffering from an affliction requiring study by PET imaging.

To overcome this limitation, the present invention provides systems and methods that can be used with PET systems to allow a tracer labeled with a pure positron emitter isotope to be combined with other tracers labeled with isotopes that emit additional gamma rays simultaneously with the positron emission. Detecting the auxiliary prompt gamma rays in coincidence or temporal correlation with the annihilation events allow the system to distinguish the photon events based on the isotopes originating the photon event. This information combined with differences in the uptake, decay, and pharmacokinetics of each radiotracer can be used to separate the information provided by each radiotracer using a conventional or slightly modified PET system.

Namely, a PET scanner can be modified in software and/or in hardware to acquire N-coincidence events (N-gamma rays arriving simultaneously to different detector elements of the scanner) and to obtain at least one of the energy, spatial, and temporal information of each of the detected single photons and, using a specific data-processing/sorting system and statistical reconstruction algorithm, a plurality of images can be reconstructed, corresponding to one of the administered radiotracers. Also, as will be described, additional energy-sensitive detectors may be integrated with the PET system to share a common time reference with the standard PET detectors included in the system.

Referring now to FIG. 2, a PET system 100 in accordance with the present invention includes an imaging hardware system 110 that includes a detector ring assembly 112 about a central axis, or bore 114. An operator work station 116 communicates through a communications link 118 with a gantry controller 120 to control operation of the imaging hardware system 110.

The detector ring assembly 112 is formed of a multitude of radiation detector units 122, which may include traditional PET detector units 122a and additional detector units 122b. Each radiation detector unit 122 produces a signal responsive to detection of a photon on communications line 124 when an event occurs. A set of acquisition circuits 126 receive the signals and produce signals indicating the event coordinates (x, y) and the total energy associated with the photons that caused the event. These signals are sent through a cable 128 to an event locator circuit 130. Each acquisition circuit 126 also produces an event detection pulse that indicates the exact moment the interaction took place. Other systems utilize sophisticated digital electronics that can also obtain this information regarding the precise instant in which the event occurred from the same signals used to obtain energy and event coordinates.

The event locator circuits 130 in some implementations, form part of a data acquisition processing system 132 that periodically samples the signals produced by the acquisition circuits 126. The data acquisition processing system 132 includes a general controller 134 that controls communications on a backplane bus 136 and on the general communications network 118. The event locator circuits 130 assemble the information regarding each valid event into a set of numbers that indicate precisely when the event took place and the position in which the event was detected. This event data packet is conveyed to a coincidence detector 138 that is also part of the data acquisition processing system 132.

The coincidence detector 138 accepts the event data packets from the event locator circuit 130 and determines if any two of them are in coincidence. Coincidence is determined by a number of factors. First, the time markers in each event data packet must be within a predetermined time window, for example, 0.5 nanoseconds or even down to picoseconds. Second, the locations indicated by the two event data packets must lie on a straight line that passes through the field of view in the scanner bore 114. Events that cannot be paired are discarded from consideration by the coincidence detector 138, but coincident event pairs are located and recorded as a coincidence data packet. This coincidence data packet, which constitutes traditional PET data, will be referred to as dataset 1.

Dataset 1 and other acquired data (that may include non-coincidence data and/or data corresponding to photon events with energy deviating from the standard 511 keV of PET imaging) are provided to a sorter 140. The function of the sorter in many traditional PET imaging systems is to receive the coincidence data packets and generate memory addresses from the coincidence data packets for the efficient storage of the coincidence data. In that context, the set of all projection rays that point in the same direction (θ) and pass through the scanner's field of view (FOV) is a complete projection, or “view”. The distance (R) between a particular projection ray and the center of the FOV locates that projection ray within the FOV. The sorter 140 counts all of the events that occur on a given projection ray (R, θ) during the scan by sorting out the coincidence data packets that indicate an event at the two detectors lying on this projection ray. The coincidence counts are organized, for example, as a set of two-dimensional arrays, one for each axial image plane, and each having as one of its dimensions the projection angle θ and the other dimension the distance R. This θ by R map of the measured events is call a histogram or, more commonly, a sinogram array. It is these sinograms that are processed to reconstruct images that indicate the number of events that took place at each image pixel location during the scan. The sorter 140 counts all events occurring along each projection ray (R, θ) and organizes them into an image data array.

In accordance with the present invention, the sorter 140 may perform the above-described functionality of a traditional PET system, but also process and sort additional data corresponding to photon events that are distinguishable from the traditional PET data set in at least one of time, number of photons emitted per decay, and energy. This additional data, which may constitute multiple datasets, will be referred to as dataset 2.

The sorter 140 provides image datasets 1 and 2 to an image processing/reconstruction system, for example, by way of a communications link 144 to be stored in an image array 146. The image arrays 146 hold the respective datasets for access by an image processor 148 that reconstructs images, at least one corresponding to one of the datasets.

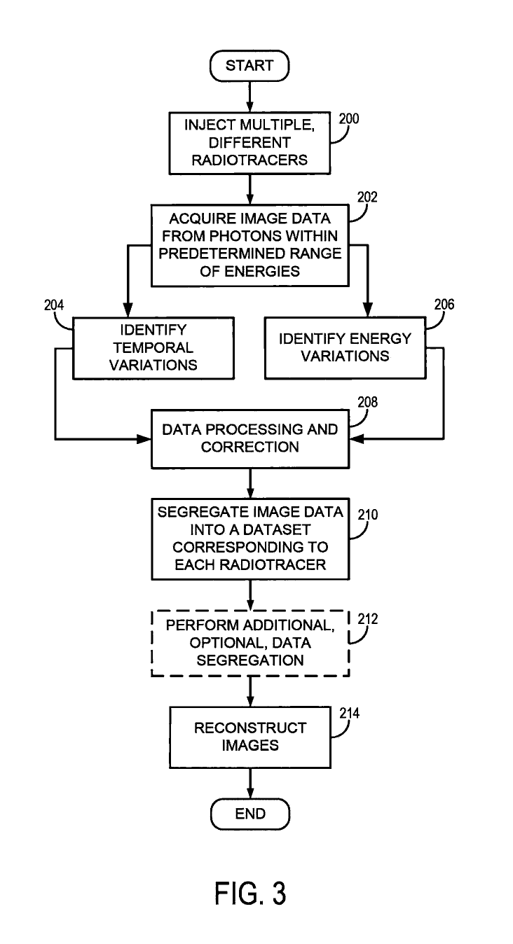

Referring now to FIG. 3, a process for acquiring image data and creating images in accordance with the present invention will be described. As described above with respect to FIG. 1, the present invention may be practiced utilizing any of a variety of available emission tomography systems, including PET or SPECT, or may utilize specialized systems specifically tailored to multiplexable emission tomography. Also, as described above with respect to FIG. 2, the present invention may include modifications to commercial imaging systems, such as a PET system. However, FIG. 3 will be described with respect to general systems and methods of the present invention and not limited to a particular commercially-available system or hardware or software modification of a particular system or method.

Specifically, referring to FIG. 3, a process for multiplexable emission tomography in accordance with the present invention begins at process block 200 with the injection of multiple different radiotracers. Each radiotracer is labeled with a selected radionuclide and, it may be introduced into the subject under study using staggered injections. The order of the injections may depend on the physical characteristics of the isotopes used to label the radiotracers and the kinetics of the selected radiotracers. One of these tracers may be labeled with a pure positron emitter and each of the remaining radiotracers may be labeled with isotopes that emit additional gamma rays in cascade with the positron emission. The energy of the additional cascade gamma rays emitted by each of those isotopes is preferably selected to be individually distinguishable between each other if the number of radiotracers injected is bigger than two.

At process block 202, image data is acquired by detecting and recording N-photon coincidences (N-photons detected within a narrow coincidence window on the order of, for example, picoseconds or nanoseconds in different detectors of the scanner) and across a predetermined range of energies. That is, a wide range of image data is collected to ensure that data for each photon event associated with each of the administered radiotracers is acquired.

As indicated at process block 204 and 206, the energy of each of the photons comprising a coincidence event and their arrival time is determined with certain precision. As illustrated, these processes may, in a process flow, occur substantially in parallel or independently or, alternatively, may be performed in series.

To perform the steps represented by process block 204, specific arrangements may be advantageous to optimize a given system for the detection of N-photon coincidences, including triple coincidences. For example, in a traditional PET system, triple coincidences are considered to represent erroneous or at least unfavorable data. However, in accordance with the present invention, such triple (or even greater) coincidences may represent desirable data and, as such, a timing calibration of the scanner for triple (or greater) coincidences may be desirable to aid in the identification of temporal variations at process block 204.

Thus, the data may be stored in a large list of events (list-mode) or in a histogram format (typically a sinogram, or line-of-response (LOR) histograms). However, unlike traditional PET imaging where N-tuples coincidences (N>2) are usually discarded and therefore not recorded, all N-tuples coincidences corresponding to N events detected within the time coincidence window may be recorded.

This information can be encoded in a more compact format, by using, for instance, the number of the LORs or sinogram bins that can be obtained from all allowed combinations of pairs of detected events. For example, one dataset can contain the standard double coincidences, each one associated to a specific LOR or sinogram bin. Additionally, another dataset may contain triples coincidences (or N-photon coincidences); each one associated to three LORs or sinogram bins. Due to geometric constraints, one or several of the possible LORs associated with a multiple photon event may lie outside of the FOV and, therefore, may be discarded as it will not correspond to a valid LOR or sinogram bin. In this case, a triple coincidence, for example, may be associated only to a predetermined number, such as two, LORs or sinogram bins.

Once the acquisition of the data has finished, or during the data acquisition, the information is processed to, ultimately, provide a series of registered, radiotracer specific images. First, at process block 208, preprocessing and data correction is performed. For example, attenuation correction for double and triple coincidences may be performed. This correction is different from standard attenuation correction performed for double coincidences in PET, as may also include a correction factor for attenuation of the third (or more) gamma ray. This can be obtained based on the information obtained from an a priori imaging acquisition, such as a separate CT acquisition. Also, it is contemplated that random estimation processing and processing to reject possible false double and triple coincidences may be performed. Of course, the processing may also include sets of standard corrections related with imaging physics (e.g. decay correction of the radioactive sources or dead time of the acquisition electronics). Further still, normalization correction may be performed to compensate for variations in the sensitivity of each detector element. This correction, takes into account that sensitivity for doubles and multiple photon coincidences are different.

At process block 210, the coincident events detected during the acquisition are classified or segregated into N different datasets, where N is the number of different radiotracers injected to the patient. A first dataset (dataset A) may contain two-photon coincidence events coming from the radiotracers injected to the patient or subject under study. The energy of the coincidence photons accepted in dataset A, for example, may be in a range that includes 511 keV. The other N−1 datasets contain events corresponding to n-photon coincidence events (being n bigger than two). Data in each of these datasets will include events in which two of the coincident photons are within the range of energy coming from positron annihilation (511 keV) and at least other gamma ray detected concurrently with them with energy coherent with the emission spectra of one of the positron-gamma emitter isotopes injected. Events in each of these datasets will not be in any of the other datasets.

At process block 212, these N datasets may be further subdivided into smaller subsets taking into account several factors of interest, like the acquisition time of each event, and/or the time-of-flight (TOF) information (difference in arrival time between each detected gamma-ray in a coincidence event). This may be done for both double and n-photon coincidences (n>2). Also, an additional classification, based on the detectors in which gamma-rays with specific properties have interacted, is also possible.

Finally, as will be described in further detail, at process block 214, a set of images is reconstructed, where at least one image is created to correspond to at least one of the administered radiotracers.

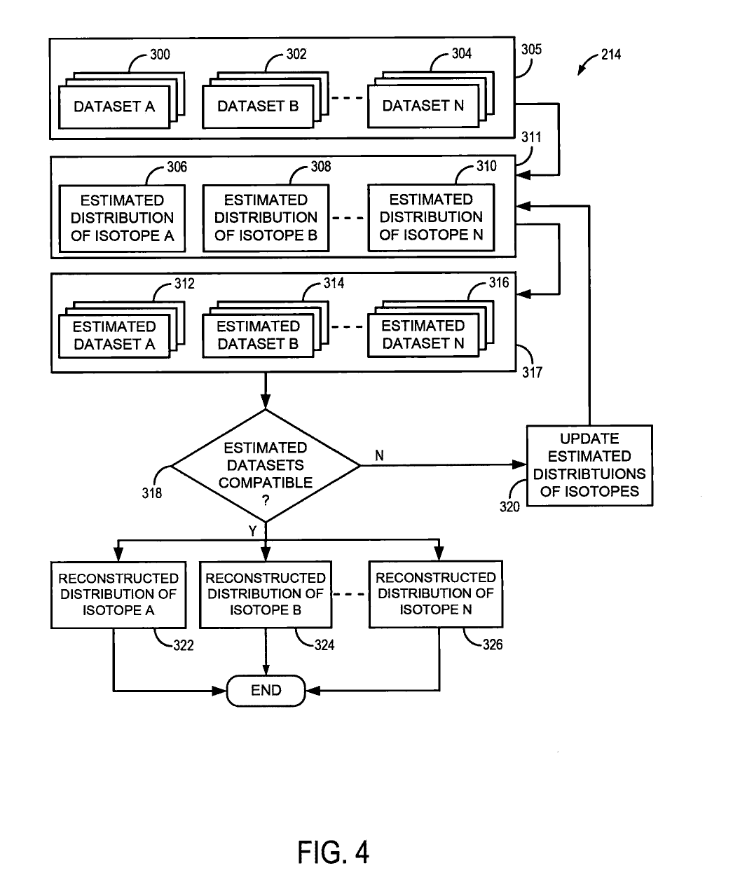

Referring now to FIG. 4, the steps of a method to reconstruct activity concentrations from multiple-radiotracer datasets, such as acquired using the methods described with respect to FIG. 3, is illustrated. To this end, the process is generally indicated by arrow 214. The above-described N datasets 300, 302, 304 represent the total data acquired 305 and may be processed using a specifically-designed reconstruction algorithm that provides N different images, for example, one for each radiotracer, that will be co-registered in space and time. These images will correspond to the most likely distribution of activity of each radiotracer that may have produced the acquired datasets 306, 308, 310, which are collectively an estimated distribution 311.

The relation between the reconstructed activity and the data acquired may be obtained using a model that takes into account the energy and arrival time of the detected gamma rays of each coincidence event, the characteristics of the gamma-ray and positron emissions of each isotope, the dynamic information (such as the evolution over time of the activity concentration in different regions of the patient or object under study), the information about the bio-distribution of the tracers obtained using kinetic modeling, the half-life of the isotopes used to label each radiotracer, time of flight information if the system is able to provide and the like.

This method may take into consideration the expectation maximization of the maximum likelihood (EM-ML) that the reconstructed radiotracer distributions have produced the measured coincidences. This method can be derived for a set of N isotopes having the above-listed estimated distribution 306, 308, 310 that are translated into estimated datasets 312, 314, 316, collectively an estimated dataset 317, using an iterative method.

For exemplary purposes, the following explanation generally refers to the use of two radiotracers, radiotracer A and radiotracer B, where radiotracer A is pure-positron emitter and radiotracer B is a positron emitter that also emits an additional gamma-ray. However, it is contemplated that any number of radiotracers can be used and the selection of a pure-positron emitter and other emitters is equally flexible. As such, the following example will discuss the acquired data being separated into two datasets with time information, namely, double coincidences D (emitted from both radiotracer A and radiotracer B), and triple coincidences T (emitted by radiotracer B). In a more general derivation, different subsets of data based on TOF information and/or the detector in which the 3rd gamma was detected may be created. In that case, an estimation of the expected number of detected coincidences for each of these cases should be obtained.

With two radiotracers and two main datasets, the estimated measurements for each dataset 312, 314, 316 can be obtained according to:

$$\begin{matrix}{λ}_{i}^{D} ( t ) = {\sum }_{j}^{ } {a}_{ij} ( {x}_{j}^{A} ( t ) + ( 1 - {β}_{j} ) {x}_{j}^{B} ( x ) ) ; & eqn . 1 \\ {λ}_{i}^{T} ( t ) = {\sum }_{j}^{ } {a}_{ij} {β}_{j} {x}_{j}^{B} ( x ) ; & eqn . 2\end{matrix}$$

where λᵢ^{D}(t) and λᵢ^{D}(t) are the estimated double and triple coincidences respectively in the line of response (LOR), i, at acquisition time t, aᵢⱼ is the probability that two gamma rays from annihilation of a positron emitted at voxel j are detected in LOR i, xⱼ^{A}(t) and xⱼ^{B}(t) are the estimated activity concentration of radiotracer A and radiotracer B respectively at acquisition time t, and βⱼ is the probability of detection of the additional gamma rays emitted by radiotracer B at voxel j.

Under these conditions, the likelihood l(x) that the reconstructed activity distributions x (from which estimations λ are generated) has emitted the measured data g (the datasets considered here gᵢ^{D} and gᵢ^{T} for double and triple coincidences respectively) is, assuming a Poisson statistics:

$$l ( x ) {\prod }_{i} \frac{{{e}^{- {λ}_{i}^{D}} ( {λ}_{i}^{D} )}^{{g}_{i}^{D}}}{{g}_{i}^{D} !} • {\prod }_{i} \frac{{{e}^{- {λ}_{i}^{T}} ( {λ}_{i}^{T} )}^{{g}_{i}^{T}}}{{g}_{i}^{T} !} . \qquad eqn . 3$$

The maximization of the likelihood is one non-limiting example of the possible criteria that can be used to implement a reconstruction algorithm. Maximization of this likelihood constrained to a non-negative solution yields an optimal solution with Kuhn-Tucker conditions for each acquisition time t:

$$\begin{matrix}{x}_{j}^{A} ( t ) • [ {\sum }_{i} {a}_{ij} • \frac{{g}_{i}^{D} ( t )}{{λ}_{i}^{D} ( t )} - {\sum }_{i} {a}_{ij} ] = 0 ; and & eqn . 4 \\ {x}_{j}^{B} ( t ) • [ {\sum }_{i} {a}_{ij} • ( ( 1 - {β}_{j} ) \frac{{g}_{i}^{D} ( t )}{{λ}_{i}^{D} ( t )} + {β}_{j} \frac{{g}_{i}^{T} ( t )}{{λ}_{i}^{T} ( t )} ) - {\sum }_{i} {a}_{ij} ] 0. & eqn . 5\end{matrix}$$

In the one implementation, this solution can be computed in an iterative fashion using one of a number of possible implementations. For example, generally speaking, the estimated datasets are compared with the data to determine whether they are statically compatible at decision block 318 and, if not, at process block 320, the estimated distributions of each radiotracer is updated. Without reducing the concept to a particular expression for the computation of this solution, one possible implementation is:

$$\begin{matrix} {x}_{j}^{A ( n + 1 )} ( t ) = {x}_{j}^{A ( n )} ( t ) • [ {\sum }_{i} {a}_{ij} • \frac{{g}_{i}^{D} ( t )}{{λ}_{i}^{D} ( t )} ] / {\sum }_{i} {a}_{ij} ; & eqn . 6 \\ {x}_{j}^{B ( n + 1 )} ( t ) = {x}_{j}^{B ( n )} ( t ) • [ {\sum }_{i} {a}_{ij} • ( ( 1 - {β}_{j} ) \frac{{g}_{i}^{D} ( t )}{{λ}_{i}^{D} ( t )} + {β}_{j} \frac{{g}_{i}^{T} ( t )}{{λ}_{i}^{T} ( t )} ) ] / {\sum }_{i} {a}_{ij} ; & eqn . 7\end{matrix}$$

where x(n) denotes the activity concentration x in the n-th iteration step. The initial estimation x⁽⁰⁾ can be selected as any smooth non-negative activity distribution. For instance, x⁽⁰⁾ may be equal to 1.0 for all voxels and isotopes considered. It is contemplated that different regularization methods that reduce the noise in the reconstructed images can be applied in this iterative reconstruction scheme. Once the datasets have been iteratively processed sufficiently, an image or images for each radiotracer is reconstructed, as illustrated by process block 322, 324, and 326.

Other example of a possible method for performing the reconstruction of images from multiplexed emission tomography (MET) data consists on the reconstruction of several independent images from each dataset (S) of the N datasets obtained, using, for example, a EM-ML algorithm (eq. 8). The appropriate regularization methods and corrections may be used to obtain the best possible image quality.

$${x}_{j}^{S ( n + 1 )} ( t ) = {x}_{j}^{S ( n )} ( t ) • [ {\sum }_{i} {a}_{ij} • \frac{{g}_{i}^{S} ( t )}{{λ}_{i}^{S} ( t )} ] / {\sum }_{i} {a}_{ij} . \qquad eqn . 8$$

Each of these images x^{S} will contain information of the aggregated distribution of all the radiotracers that contributed to generate the corresponding dataset S. These images may be further processed to obtain images of the distribution of each radiotracer. The separation process may consist on analytical or iterative methods. For example, an EM-ML algorithm can be employed for that purpose.

For exemplary purposes, the same case of described above may be considered. In that case, two images x^{D} and x^{T} can be reconstructed, from the doubles and triples datasets respectively. The doubles dataset D contain information from the two radiotracers (A and B) considered, while the triples dataset T only contains information of the radiotracer B. The images of isotope A and B may be obtained iteratively, starting with a uniform distribution and updating in the iteration n+1 by:

$${x}_{j}^{A ( n + 1 )} ( t ) = {x}_{j}^{A ( n )} ( t ) \cdot [ \frac{{x}_{j}^{D} ( t )}{{x}_{j}^{A} ( t ) + {x}_{j}^{B} ( t )} ] \\ {x}_{j}^{B ( n + 1 )} ( t ) = {x}_{j}^{B ( n )} ( t ) \cdot [ ( 1 - α ) \cdot \frac{{x}_{j}^{D} ( t )}{{x}_{j}^{A} ( t ) + {x}_{j}^{B} ( t )} + α \frac{{x}_{j}^{T} ( t )}{{β}_{j} {x}_{j}^{B} ( t )} ] . \qquad eqn . 9$$

The parameter α controls the relative weight of the Triple dataset in the estimation of the distribution of isotope B. It can be chosen to be βⱼ but other weights may be used. Different regularization methods can be used during the reconstruction, including the use of some a priori information about the local level of smoothness to avoid the excess of noise in the final images.

The presence of the temporal information allows a better isotope separation. In many cases, one of the isotopes will have a slow variation with time, while the other will have a significant change along time during the acquisition. These differences may be due to the different half-life of the isotopes and/or by the kinetics of the injected radiotracers.

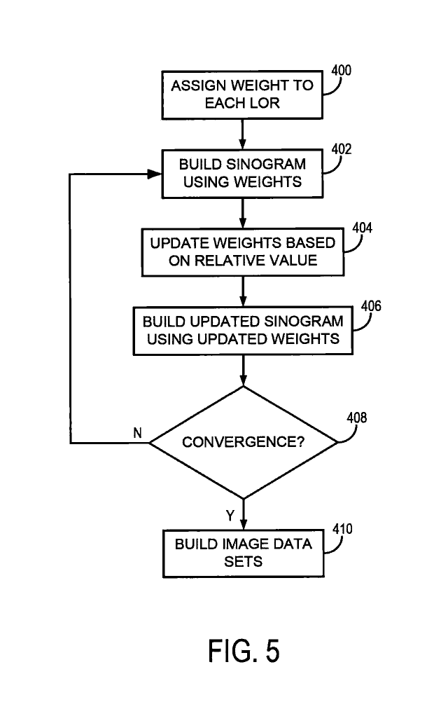

Referring to FIG. 5, a flow chart setting forth the steps of one non-limiting example of a method for processing triple coincidences as part of a reconstruction process, such as reconstruction process 214 of FIG. 3, is provided. The process begins at process block 400 where each LOR of the n-coincidence is assigned a weight. A given triple coincidence, “i”, is composed of three LORs, each one with weights wᵢ₁, wᵢ₂, and wᵢ₃, where the sum of wᵢ₁, wᵢ₂, and wᵢ₃ is 1, such that:

Tripleᵢ={(wᵢ₁,LORᵢ₁),(wᵢ₂,LORᵢ₂)(wᵢ₃,LORᵢ₃)}, i=1 . . . N_{Triples} eqn. 9

If no additional information is available, wᵢ₁, wⁱ², and wᵢ₃ are set to the same weight (⅓). If one of the LORs, for instance LORᵢ₃, is not valid due to geometrical reasons, wᵢ₁=wᵢ₂ are set to ½ and wᵢ₃ is set to 0. These weights represent the probability of each of these lines to be the true line of response.

At process block 402, using the weights in each LOR, a sinogram (or in other similar format like a LOR histogram) can be created. Then, the number of counts in each bin of this sinogram can be used to modify the weights at process block 404. For instance, one possible method is modifying the weights according to the following equation:

$${w}_{ij} = \frac{{N}_{ij}}{{N}_{i 1} + {N}_{i 2} + {N}_{i 3}} , i = 1 … {N}_{Triples}$$

where Nᵢⱼ represents the occurrence rate for the LORᵢⱼ in the sinogram. Other criteria for the calculation of the weights are also within the scope of the present disclosure.

After all the weights have been updated, a new sinogram is created based on the new weights at process block 406. At decision block 408, the data are reviewed for convergence and the procedure is repeated until convergence criterion or criteria are reached. At process block 410, the image data sets are built. Specifically, the sinogram obtained using the final weights wᵢⱼ represents the activity distribution of the radionuclide emitting the multiple photons, such as triple coincidences in this non-limiting example.

The systems and methods described herein provide improvements over previously proposed multi-isotope or multi-tracer imaging techniques. For example, so-called attempts at dual-isotope PET imaging technique proposed the use of two different isotopes during imaging using a traditional PET system. One of the isotopes is a pure positron emitter and the other isotope emits additional gamma rays together with the positron emission. The acquired data was proposed to be separated into a dataset of two gamma-ray coincidences (A) and a dataset of three gamma-ray coincidences (B). The image obtained from reconstructing dataset B is used to estimate the amount of each tracer in dataset A (See, A Andreyev and A Celler, “Dual-isotope PET using positron-gamma emitters,” Physics in Medicine and Biology 56, no. 14 (Jul. 21, 2011) 4539-4556). Unfortunately, this technique does not consider dynamic or temporal information, which limits its applicability and leads to the production of images with considerably lower quality in terms of noise, even when compared to current PET images. In the Medical Imaging Conference, IEEE November 2012, Andreyev included temporal information. (EM Reconstruction of Dual Isotope PET with Staggered Injections and Prompt Gamma Positron Emitters A. Andreyev, A. Sitek, A. Celler). However, the method proposed in those works requires the energy of the prompt gamma ray emitted by the positron-gamma emitter to be distinguishable from 511 keV Notably, some positron gamma emitters such as ¹²⁴I or ⁷⁶Br may have energies too close to 511 keV and, thus, it would be difficult to distinguish the radiotracers by energy with the energy resolution of most common PET scanners. Instead, the present invention can distinguish such data using the different number of emissions, such as triples or more. Thus, the present invention can distinguish the datasets even without energy separation.

Another existing technique, named “multi-tracer PET,” uses staggered injections of two different tracers labeled with the same or different isotopes to obtain separated images of each tracer based only in the differences in the kinetics of each tracer. (See, for example, Noel F Black, Scott McJames, et al., “Evaluation of rapid dual-tracer 62 Cu-PTSM+62Cu-ATSM PET in dogs with spontaneously occurring tumors,” Physics in Medicine and Biology 53, no. 1 (Jan. 7, 2008): 217-232; D. J Kadrmas and T. C Rust, “Feasibility of rapid multitracer PET tumor imaging,” IEEE Transactions on Nuclear Science 52, no. 5 (October 2005): 1341-1347; N. F Black, S. McJames, and D. J Kadrmas, “Rapid Multi-Tracer PET Tumor Imaging With Secondary Shorter-Lived Tracers,” IEEE Transactions on Nuclear Science 56, no. 5 (October 2009): 2750-2758; and Dan J. Kadrmas et al., “RAPID MULTI-TRACER PET IMAGING SYSTEMS AND METHODS”, Sep. 25, 2008, US Publication No. 2008/0230703). “Rapid Multi-Tracer PET Using Reduced Parameter Space Kinetic Modeling” D. J. Kadrmas, M. B. Oktay—MIC IEEE 2012, “Single-scan dual-tracer FLT+FDG PET tumor characterization” Dan J Kadrmas, Thomas C Rust, and John M Hoffman, Phys Med Biol. 2013 Feb. 7; 58(3): 429-449. In this technique differences in decay among tracers and dynamic information are used, but signal separation is based on tracer-dependent kinetic models that have large uncertainties. As such, the accuracy of the method is limited. On the other hand these methods are not oriented to obtain at least one image corresponding to at least one of the injected radiotracers, but are intended to estimate parameters of a kinetic model.

The systems and methods of the present invention, instead, provide multiplexed emission tomography, which overcomes the limitations of the aforementioned methods by combining temporal discrimination and energy discrimination and a tailored reconstruction algorithm that can take into account the characteristics of the positron and gamma-ray emissions of each tracer, dynamic information about the radiotracer distribution, information about the bio-distribution of the tracers obtained from kinetic modeling, the half-life of the isotopes used to label each radiotracer, and TOF information the system is capable of providing. As such, the systems and methods of the present invention provide higher signal-to-noise ratio (SNR) and better quantification accuracy than the previous methods.

In addition, multi-isotope, multiplexable imaging in accordance with the present invention is readily compatible with SPECT systems. Each radiotracer is labeled with a different gamma emitter isotope that emits a characteristic gamma-ray with a known energy. Signal separation is done based on the energy differences of the detected gamma rays. Notably, some radiotracers may have energies too close to 511 keV and, thus, it would be difficult to distinguish the radiotracers by energy with the energy resolution of most common PET scanners. Instead, the present invention can distinguish such data using the different number of emissions, such as triples or more. Thus, the present invention can distinguish the datasets even without energy separation.

A similar approach for multiplexed emission tomography using SPECT and radiotracer labeled with a positron emitter isotope and other radiotracers labeled with a gamma ray emitter isotope scanner have been previously proposed. Unfortunately, these systems and method suffer from various drawbacks. One problem in these systems and methods is the contamination of the data from the gamma emitter isotope due to the high-energy gamma rays emitted from the positron emitter isotope. Another limitation of these techniques using SPECT is their low sensitivity (100 to 1000 times lower than PET) and low spatial resolution (this parameter is typically 5 mm with clinical PET systems and 10 to 14 mm with SPECT). Thus, the present invention's compatibility with PET systems not only provides the inherent higher quality over the use of SPECT system, but can be accurately quantified in terms of local activity concentration (MBq/ml).

The present invention had broad applicability. For example, systems and methods of the present invention are useful in preclinical applications. In such settings, the present invention can provide unique opportunities for determining inter-linked biologic parameters in-vivo with better sensitivity (from ×20 up to ×200) than it is currently possible to achieve (e.g., using dual isotope SPECT). It also provides new research possibilities in oncology, neurology and cardiology opening a broad range of alternatives that can be tested in preclinical studies prior to its clinical adoption.

Of course, the present invention also has broad applicability to clinical applications. In general, the possibility of performing true, simultaneous, multiple-tracer PET acquisitions increases the effective number of studies that can be acquired in scanner, providing an important reduction in imaging time and costs and the further advantages of automatic image registration.

Furthermore, the present invention provides better information to radiotherapy planning than traditional methods. External beam radiation therapy procedures have, until recently, been planned almost exclusively using anatomic imaging methods. Molecular imaging using hybrid PET/computed tomography scanning has provided new insights into the precise location of tumors and the extent and character of the biologically active tumor volume and has provided differential response information during and after therapy. In addition to the commonly used radiotracer ¹⁸F-FDG, additional radiopharmaceuticals are being explored to image major physiological processes, as well as tumor biological properties, such as hypoxia, proliferation, amino acid accumulation, apoptosis, and receptor expression. This provides the potential, along with the systems and methods of the present invention, to target or boost the radiation dose to a biologically relevant region within a tumor, such as the most hypoxic or most proliferative area. Accurate image registration between the functional image set and the treatment planning set is key to ensuring a dosimetric benefit. Inaccurate image registration can, in the worst case, result in the opposite effect being achieved (i.e., a higher dose to more functional normal tissue regions). The present invention enables a clinical PET/CT scanner to acquire in a single session anatomical information (CT) and information about several physiological parameters of tumors (i.e., glucose metabolism, hypoxia) for radiation therapy planning. The anatomical information and the glucose image can be used to delineate the active area/s of the tumor/s and the joint information from glucose metabolism and hypoxia, which is related to tumor resistance to radiation, can be used to modulate the radiation dose which will be delivered to different areas of the tumor. In other words, information of FDG and a hypoxia tracer together with CT, allows planning a radiation dose delivery proportional to the resistance of the tissue, optimizing therapy for cancerous tissues and respecting healthy tissues as convenient.

The present invention provides an additional mechanism to reduce the dose received by some nuclear medicine patients. In patients who currently need several separate PET-CT acquisitions to measure, for example, myocardium viability with PET, corresponding CTs for each acquisition are used to calculate attenuation maps. The total amount of dose for the patient will be reduced using the present invention, as only one CT scan corresponding to the one PET acquisition will be needed. Under such clinical protocols, the CT imaging is responsible for the largest amount of dose delivered to a patient and the limiting thereof is a substantial clinical advantage.

The present invention can also improve the detection of cancerous lesions. FDG-PET is an effective but imperfect tool for cancer detection and staging that takes advantage of a common defect in tumor metabolism: inefficient and elevated glucose consumption. A combination of FDG-PET with other tracers to probe alternative metabolic pathways, tumor characteristics or receptors in the tumor cells has proven to improve sensitivity and specificity in difficult cases such as neuroendocrine tumors, certain lung tumors, hepatocellular carcinoma and liver tumors, brain tumors, colon tumors, and intrapelvic tumors, among others.

The present invention can also be used to predict and monitor therapy response. For example, several studies have combined PET imaging of blood flow and glucose metabolism to predict and measure response to neoadjuvant chemotherapy in breast cancer patients. The ratio of low glucose metabolism to high blood flow was found to be the best predictor of a positive response to treatment, and also predicted longer disease-free survival. This prediction could not be made with measurement of glucose metabolism alone since treatment responders show only a slightly greater reduction in glucose metabolism as compared with non-responders.

Further still, the present invention can be used to improve tumor characterization and select the best treatment for each patient. For example, hypoxia is a critical factor in carcinogenesis, and hypoxic tumors are more resistant to both radiation and chemotherapy than tumors that are not hypoxic. Variations in hypoxia and glucose metabolism have been studied in a variety of human tumors using PET. These variations were considered to reflect ubiquitous genetic responses to hypoxic stress. The complementary information provided by both parameters is considered could allow simultaneous diagnosis or staging of disease and treatment selection for each specific case.

The present invention has been described in terms of one or more preferred embodiments, and it should be appreciated that many equivalents, alternatives, variations, and modifications, aside from those expressly stated, are possible and within the scope of the invention. Therefore, the invention should not be limited to a particular described embodiment.

1. An emission tomography system for acquiring a series of medical images of a subject during a common imaging process using multiple radiotracers, the system comprising:
a plurality of detectors configured to be arranged about the subject to acquire gamma rays emitted from the subject as a result of multiple radiotracers administered to the subject and communicate signals corresponding to acquired gamma rays;
a data processing system configured to receive the signals from the plurality of detectors, identify temporal information and energy information of photons of the acquired gamma rays, and distinguish signals from at least one of the multiple radiotracers, wherein the temporal information is identified with sufficient temporal resolution to determine coincidence events; and
a reconstruction system configured to receive the signals, the temporal information, and the energy information from the data processing system and reconstruct therefrom a series of medical images of the subject, wherein at least one of the images in the series of medical images corresponds to only information acquired from gamma rays emitted as a result of a given one of the multiple radiotracers.

2. The system of claim 1 wherein the plurality of detectors are configured to have a temporal resolution of at least one of nanosecond and picosecond.

3. The system of claim 2 wherein the data processing system is configured to determine the coincidence events with respect to the at least one of the nanosecond and the picosecond temporal resolution.

4. The system of claim 1 further comprising a plurality of energy-sensitive detectors configured to share a common time reference with the plurality of detectors and generate energy-sensitive signals in response to detected photons.

5. The system of claim 1 wherein at least one of the data processing system and the reconstruction system is configured to apply a normalization to the signals from the plurality of detectors.

6. The system of claim 1 further comprising a number of image arrays corresponding to a number of distinct radiotracers administered to the subject and wherein each image array is configured to store respective datasets associated with each distinct radiotracer for access by the reconstruction system to reconstruct one image corresponding to each radiotracer.

7. A method for acquiring a series of medical images of a subject having been administered at least two radiotracers selected to emit photons distinguishable in at least one of time and energy, the method comprising:
detecting, during an imaging process, photons emitted from the subject as a result of the at least two radiotracers administered to the subject;
creating imaging data based on the detected photons;
processing the imaging data to identify temporal information including coincidence events and energy information associated with the detected photons;
sorting the imaging data into datasets distinguished by at least one of the temporal information and the energy information, wherein at least one dataset corresponds to only one of the at least two radiotracers;
reconstructing a series of medical images of the subject, wherein at least one of the images in the series of medical images corresponds to only one of the at least two radiotracers.

8. The method of claim 7 further comprising using a positron emission system (PET) imaging system to detect the photons.

9. The method of claim 8 wherein the PET imaging system is configured to operate as a multiplexed emission tomography system (MET) imaging system.

10. The method of claim 7 wherein at least one of the two radiotracers is labeled with a pure positron emitter radionuclide and at least one of the two radiotracers is labeled with a radionuclide that emit additional gamma rays simultaneously with the positron emission.

11. The method of claim 7 wherein sorting includes separating data in the imaging dataset based on at least one of differences in an expected uptake between the at least two radiotracers, an expected decay of the at least two radiotracers, and an expected pharmacokinetics of the at least two radiotracers.

12. The method of claim 7 wherein sorting includes separating data in the imaging dataset based on differences in a number of photons emitted per radioactive decay of the at least two radiotracers.

13. The method of claim 7 wherein separated images corresponding to each of the radiotracers are generated using reconstructed images corresponding to each of the sorted datasets.

14. The method of claim 13 wherein an iterative method is used to separate images corresponding to each of the radiotracers.

15. The method of claim 7 wherein an iterative method is used to reconstruct images corresponding to each of the sorted datasets.

16. The method of claim 7 further comprising performing attenuation correction for at least one of double coincidences and triple coincidences.

17. The method of claim 16 wherein the attenuation correction includes a correction factor for attenuation of prompt gamma rays.

18. The method of claim 17 wherein correction factor is based on information obtained from an a priori imaging acquisition.

19. The method of claim 7 further comprising applying a normalization correction to the imaging data, wherein the normalization correction is based on at least one of sensitivity of the imaging data and energy of the photons associated with the imaging data.

20. The method of claim 7 further comprising applying a normalization correction to the sorted datasets, wherein the normalization correction is based on at least one of sensitivity of the sorted datasets and energy of the photons associated with the sorted datasets.

21. The method of claim 20 wherein a different normalization correction is applied to each sorted dataset, wherein the normalization correction is based on at least one of sensitivity of each sorted dataset and energy of the photons associated with each sorted dataset.

22. The method of claim 7 wherein sorting the imaging data includes using a model that takes into account at least one of the dynamic information including evolution over time of activity concentration in different regions of the subject, half-life information about isotopes of the at least two radiotracers, and time-of-flight information.

23. A method for acquiring a series of medical images of a subject having been administered at least two radiotracers selected to emit photons distinguishable in at least one of time and energy, the method comprising:
acquiring, during a single scanning session, photons emitted from the subject as a result of the at least two radiotracers administered to the subject, wherein the acquired photons are selected from a predetermined energy range;
creating, based on the acquired photons, imaging data sets, wherein each imaging data set is differentiated based on temporal information including coincidence events and energy information associated with the acquired photons;
reconstructing a series of medical images of the subject from the imaging data sets, wherein at least one of the images in the series of medical images corresponds to only one of the at least two radiotracers.

24. The method of claim 23 wherein at least one of the two radiotracers is labeled with a pure positron emitter isotope and at least one of the two radiotracers is labeled with isotopes that emit additional gamma rays simultaneously with the positron emission.

25. The method of claim 23 wherein creating the imaging data sets includes identifying double coincidence events and triple coincidence events.

26. The method of claim 23 further comprising building sinograms for each of the imaging data sets.

27. The method of claim 23 wherein the sinograms are weighted using an iterative process to differentiate background information from information acquired from the subject.
